(19) United States
(12) Patent Application Publication
(10) Pub. No.: US 2020/0182688 A1
(43) Pub. Date: Jun. 11, 2020

(54) OPTICAL SENSOR DEVICE AND ELECTRONIC DEVICE INCLUDING THE SAME

(71) Applicant: Samsung Electronics Co., Ltd., Gyeonggi-do (KR)

(72) Inventors: Jeonggyu JO, Gyeonggi-do (KR); Hyeongsoon PARK, Gyeonggi-do (KR)

(21) Appl. No.: 16/708,742

(22) Filed: Dec. 10, 2019

(30) Foreign Application Priority Data
Dec. 11, 2018 (KR) 10-2018-0159143

(51) Int. Cl. G01J 1/04; G02B 3/08

(52) U.S. Cl. CPC G01J 1/0411; A61B 3/14; G01J 1/0477; G02B 3/08

(57) ABSTRACT

An electronic device is disclosed herein, including: a substrate, an optical sensor device including at least one light-emitting element and a light-receiving element, the optical sensor device mounted on the substrate, and an injection-molded lens coupled to the substrate and covering the optical sensor device, wherein the injection-molded lens is spaced apart from the optical sensor device by a set distance, wherein patterns are integrally formed in at least a portion of the injection-molded lens, the patterns affecting transmission of light of at least one wavelength band to improve an optical efficiency of the transmitted light.

### CROSS-REFERENCE TO RELATED APPLICATION(S)

This application is based on and claims priority under 35 U.S.C. 119 to Korean Patent Application No. 10-2018-0159143, filed on December 11, 2018, in the Korean Intellectual Property Office, the disclosure of which is herein incorporated by reference in its entirety.

### BACKGROUND

### 1) Technical Field

The disclosure relates to an electronic device including light emitters and light detecting sensors, and, more particularly, to disposition of patterns in a cover of the electronic device to affect the optics of light transmitted and received by the emitters and sensors.

### 2) Description of Related Art

An electronic device may perform various functions in combination, among which include functions which utilize sensor devices. The sensor devices may collect information related to the electronic device, an external environment of the electronic device, or a user. The electronic device may provide various functions or services to the user based on the information collected using the sensor devices.

For example, the electronic device may include an optical sensor device including at least one light-emitting element and a light-receiving element. The light sensor device may measure information relevant to the proximity of an object, and/or a heart rate, blood pressure, or blood sugar of a user of the electronic device, using transmitting and reception of light of a specific wavelength band.

The above information is presented as background information only to assist with an understanding of the disclosure. No determination has been made, and no assertion is made, as to whether any of the above might be applicable as prior art with regard to the disclosure.

SUMMARY

The light sensor device may have a relatively low thickness compared to other electronic components included in the electronic device. As an air gap increases, optical efficiency may be lowered. For this reason, in order to mount the light sensor device in the electronic device, it is desirable to utilize an interposer for minimizing energy loss caused by the air gap, which may compensate for a height difference with another electronic component. However, this may introduce a problem in production steps and/or unit costs may increase, due to the addition of the interposer for mounting the light sensor device.

In addition, due to the diversification of services, the light sensor device frequently utilizes various light-emitting elements having different wavelength bands, even as the area allocated for disposition of the light sensor device increases. As the area of the light sensor device increases, the light sensor device is increasingly exposed to the external environment of the electronic device, and thus the aesthetic appeal of the electronic device deteriorates. Accordingly, in the electronic device, there is a need for improving the design of the electronic device while maintaining and/or improving optical efficiency, and compensating for the air gap without the drawbacks of the interposer structure.

An electronic device according to certain embodiments may include: a substrate, an optical sensor device including at least one light-emitting element and a light-receiving element, the optical sensor device mounted on the substrate, and an injection-molded lens coupled to the substrate and covering the optical sensor device, wherein the injection-molded lens is spaced apart from the optical sensor device by a set distance, wherein patterns are integrally formed in at least a portion of the injection-molded lens, the patterns affecting transmission of light of at least one wavelength band to improve an optical efficiency of the transmitted light.

An electronic device according to certain embodiments may include: a circuit board including a substrate, an optical sensor device including at least one light-emitting element and a light-receiving element, the optical sensor device mounted on the substrate, and an injection-molded lens coupled to the substrate and covering the optical sensor device, wherein the injection-molded lens is spaced apart from the optical sensor device by a set distance, wherein the injection-molded lens includes a top part disposed above a top face of the optical sensor device, and a side part supporting the top part as a side face of the optical sensor device, and patterns are integrally formed in at least a portion of the injection-molded lens, the patterns affection transmission of light of at least one wavelength band to improve an optical efficiency of the transmitted light.

According to certain embodiments, an injection-molded lens having a pattern integrally formed therein to improve optical efficiency for at least one wavelength is mounted in an electronic device as a structure of covering an optical sensor device so as to compensate for a height difference between the light sensor device and another electronic component. Thus, the injection-molded lens is capable of satisfying a design requirement and improving visibility.

### BRIEF DESCRIPTION OF THE DRAWINGS

For a more complete understanding of the disclosure and its features, reference is now made to the following description taken in conjunction with the accompanying drawings, in which like reference numerals represent like parts:

FIG. 1 is a block diagram illustrating an electronic device in a network environment according to certain embodiments;

FIG. 2A is a perspective view of the front surface of a mobile electronic device according to an embodiment, and FIG. 2B is a perspective view of the rear surface of the electronic device of FIG. 1; and FIG. 2C illustrates the structure of the light sensor device.

FIG. 3A is a front perspective view of an electronic device 300 according to certain embodiments, and FIG. 3B is a rear perspective view of the electronic device 300 according to certain embodiments;

FIG. 4 is a vertical cross-sectional view illustrating an optical sensor device according to certain embodiments;

FIG. 5A is a vertical cross-sectional view illustrating an optical sensor device according to certain embodiments, and FIG. 5B is a vertical cross-sectional view illustrating an optical sensor device according to certain embodiments;

FIG. 6 is a vertical cross-sectional view illustrating an optical sensor device according to certain embodiments.

### DETAILED DESCRIPTION

The electronic device according to certain embodiments may be one of various types of electronic devices. The electronic devices may include, for example, a portable communication device (e.g., a smart phone), a computer device, a portable multimedia device, a portable medical device, a camera, a wearable device, or a home appliance. According to an embodiment of the disclosure, the electronic devices are not limited to those described above.

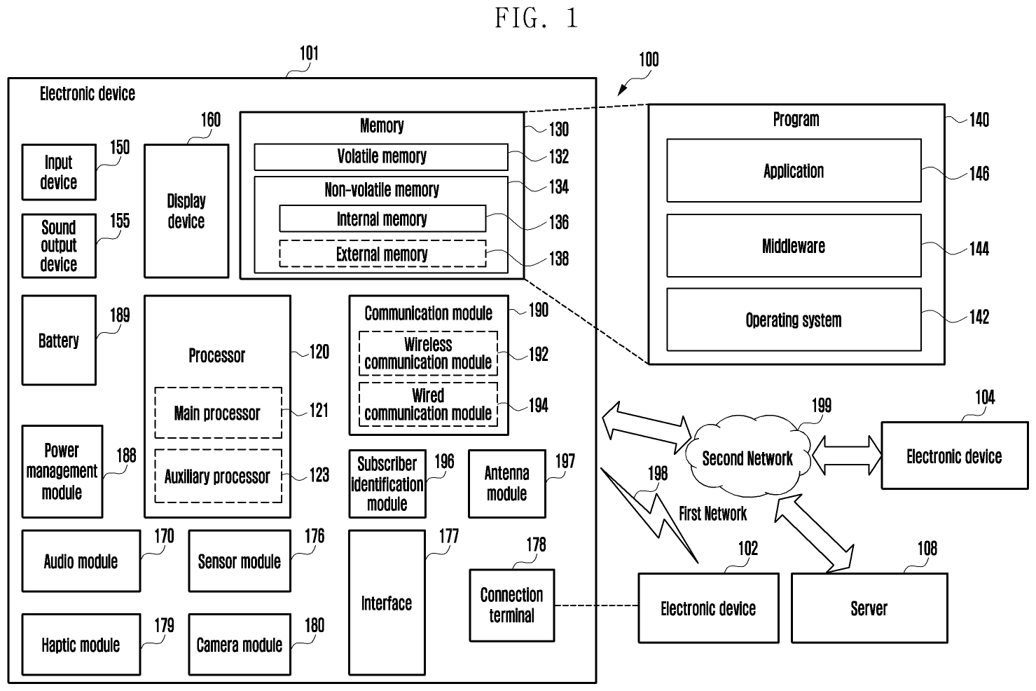

FIG. 1 is a block diagram illustrating an electronic device 101 in a network environment 100 according to certain embodiments. Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, generated via external interaction (e.g., a user input) of the electronic device 101. The input device 150 may include, for example, a microphone, a mouse, or a keyboard.

The sound output device 155 may output sound signals to the external environment of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the external environment (e.g., displaying information to a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector),

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from external devices in communication with the electronic device 101. According to an embodiment, the antenna module 197 may include one or more antennas, and, therefrom, at least one antenna appropriate for a communication scheme used in the communication network, such as the first network 198 or the second network 199, may be selected, for example, by the communication module 190 (e.g., the wireless communication module 192). The signal or the power may then be transmitted or received between the communication module 190 and the external electronic device via the selected at least one antenna.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

According to some embodiments, the sensor module 176 may obtain external environmental information or biometric information. The electronic device may detect at least one of humidity, temperature, calorie, illuminance, light, ion, vibration, radiation, sound wave, ultrasonic wave, pressure, chemical component, or biological reaction using the sensor module 176. The electronic device may detect at least one of a heart rate, fingerprint, iris, brain wave, face, blood pressure, or blood sugar using the sensor module 176.

According to some embodiments, the sensor module 176 may include an illumination sensor that incorporates a proximity sensor, a color sensor (e.g., an RGB (red, green, blue) sensor), and an infrared (IR) sensor, a biometric sensor, a heart rate monitor (HRM) sensor, a photoplethysmography (PPG) sensor, and an iris recognition sensor. According to some embodiments, the electronic device 101 may include at least one of the above-described sensors.

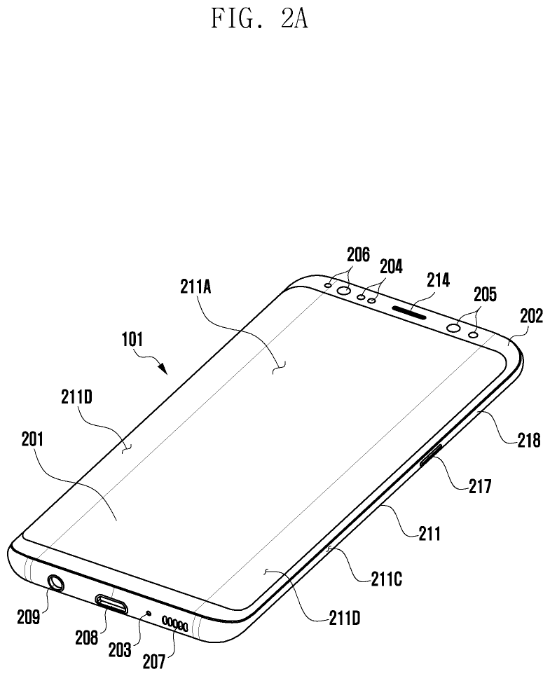

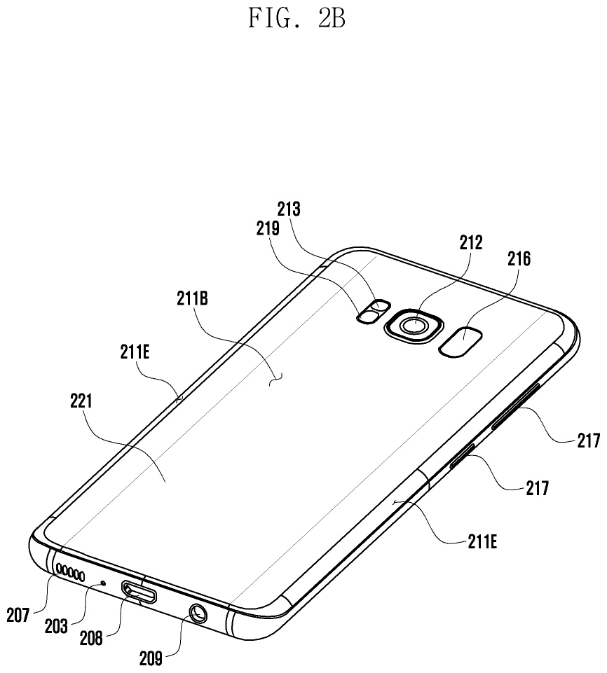

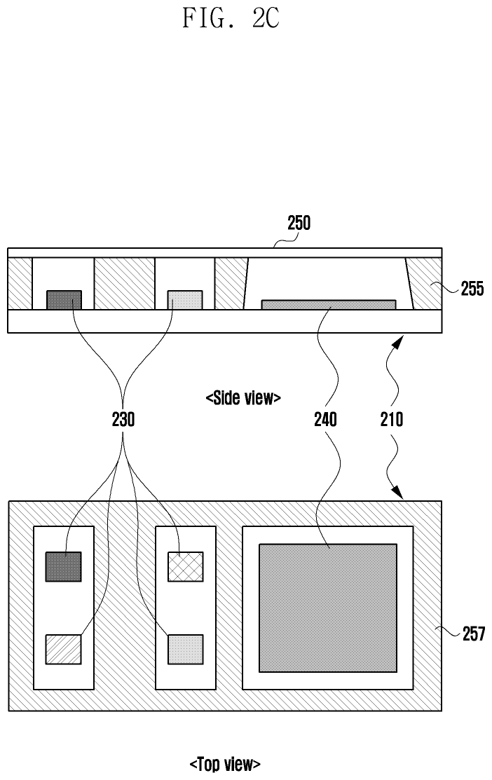

FIG. 2A is a perspective view of the front surface of a mobile electronic device according to an embodiment.

FIG. 2B is a perspective view of the rear surface of the electronic device of FIG. 1.

Referring to FIG. 2A and FIG. 2B, According to an embodiment, the electronic device 101 may include the light sensor device 210For example, the electronic device 101 may include at least one of a smart phone, a tablet personal computer (PC), a head-mounted-device (HMD((e.g., VR(virtual reality) device, AR(augmented reality) device, MR(mixed reality device, or electronic eyeglasses), electronic clothing, an electronic bracelet, an electronic necklace, an appcessory, an electronic tattoo, or a smart watch.

An electronic device 101 according to an embodiment may include a housing 211 including a first surface (or front surface) 211A, a second surface (or rear surface) 211B, and a side surface 211C surrounding the space between the first surface 211A and the second surface 211B. In another embodiment (not illustrated), the housing may denote a structure that forms a part of the first surface 211A, the second surface 211B, and the side surface 211C illustrated in FIG. 1. According to an embodiment, the first surface 211A may be formed by a front plate 202, at least a part of which is substantially transparent (for example, a glass plate including various coating layers, or a polymer plate). The second surface 211B may be formed by a rear plate 221 that is substantially opaque. The rear plate 221 may be made of coated or colored glass, ceramic, polymer, metal (for example, aluminum, stainless steel (STS), or magnesium), or a combination of at least two of the above-mentioned materials.

The side surface 221C may be formed by a side bezel structure (or “side member”) 218 which is coupled to the front plate 202 and to the rear plate 221, and which includes metal and/or polymer. In some embodiments, the rear plate 111 and the side bezel structure 218 may be formed integrally and may include the same material (for example, a metal material such as aluminum).

In the illustrated embodiment, the front plate 202 may include two first areas 211D on both ends of the long edge of the front plate 202 such that the two first areas 211D bend from the first surface 221A toward the rear plate 221 and extend seamlessly. In the illustrated embodiment (see FIG. 2a), the rear plate 221 may include two second areas 211E on both ends of the long edge such that the two second areas 211 bend from the second surface 211B toward the front plate 202 and extend seamlessly.

In some embodiments, the front plate 202 (or the rear plate 221) may include one of the first areas 211D (or the second areas 211E). In another embodiment, a part of the first areas 211D or the second areas 211E may not be included. In the above embodiments, when seen from the side surface of the electronic device 101, the side bezel structure 218 may have a first thickness (or width) on a part of the side surface, which does not include the first areas 211D or the second areas 211E as described above, and may have a second thickness that is smaller than the first thickness on a part of the side surface, which includes the first areas 1211D or the second areas 211E.

According to an embodiment, the electronic device 101 may include at least one of a display 201, audio modules as operable through the holes 203, 207, and 214), sensor modules 204, 216, and 219, camera modules 205, 212, and 213, a key input device 217, a light-emitting element 206, and connector holes 208 and 209. In some embodiments, at least one of the constituent elements (for example, the key input device 117 or the light-emitting element 206) of the electronic device 101 may be omitted, or the electronic device 101 may additionally include another constituent element.

The display 201 may be exposed through a corresponding part of the front plate 202, for example. In some embodiments, at least a part of the display 201 may be exposed through the front plate 202 that forms the first areas 211D of the side surface 211C and the first surface 1211A. In some embodiments, the display 201 may have a corner formed in substantially the same shape as that of the adjacent outer periphery of the front plate 202. In another embodiment (not illustrated), in order to increase the area of exposure of the display 201, the interval between the outer periphery of the display 201 and the outer periphery of the front plate 202 may be formed to be substantially identical.

In another embodiment (not illustrated), a recess or an opening may be formed in a part of the screen display area of the display 201, and at least one of an audio module coupled to a hole 214, a sensor module 204, a camera module 205, and a light-emitting element 206 may be included and aligned with the recess or the opening. In another embodiment (not illustrated), on the back surface of the screen display area of the display 201, at least one of an audio module coupled to a hole 214, a sensor module 204, a camera module 205, a fingerprint sensor 216, and a light-emitting element 206 may be included. In another embodiment (not illustrated), the display 201 may be coupled to or arranged adjacent to a touch sensing circuit, a pressure sensor capable of measuring the intensity (pressure) of a touch, and/or a digitizer that detects a magnetic field-type stylus pen. In some embodiments, at least a part of the sensor modules 204 and 219 and/or at least a part of the key input device 217 may be arranged in the first areas 211D and/or the second areas 211E.

The audio modules may interoperate with a microphone hole 203 and speaker holes 207 and 214. A microphone for acquiring an external sound may be arranged in the microphone hole 203, and a plurality of microphones may be arranged therein such that the direction of a sound can be sensed in some embodiments. The speaker holes 207 and 214 may include an outer speaker hole 207 and a speech receiver hole 214. In some embodiments, the speaker holes 207 and 214 and the microphone hole 203 may be implemented as a single hole, or a speaker may be included (for example, a piezoelectric speaker) without the speaker holes 207 and 214.

The camera modules 205, 212, and 213 may include a first camera device 205 arranged on the first surface 211A of the electronic device 101, a second camera device 212 arranged on the second surface 211B thereof, and/or a flash 213. The camera devices 205 and 212 may include a single lens or a plurality of lenses, an image sensor, and/or an image signal processor. The flash 213 may include, for example, a light-emitting diode or a xenon lamp. In some embodiments, two or more lenses (an infrared camera, a wide-angle lens, and a telephoto lens) and image sensors may be arranged on a single surface of the electronic device 101.

The key input device 217 may be arranged on the side surface 211C of the housing 211. In another embodiment, the electronic device 101 may not include a part of the above-mentioned key input device 217 or the entire key input device 217, and the key input device 217 (not included) may be implemented in another type, such as a soft key, on the display 201. In some embodiments, the key input device may include a sensor module 216 arranged on the second surface 211B of the housing 211.

The light-emitting element 206 may be arranged on the first surface 211A of the housing 211, for example. The light-emitting element 206 may provide information regarding the condition of the electronic device 101 in a light type, for example. In another embodiment, the light-emitting element 206 may provide a light source that interworks with operation of the camera module 205, for example. The light-emitting element 106 may include, for example, an LED, an IR LED, and a xenon lamp.

The connector holes 208 and 209 may include a first connector hole 208 capable of containing a connector (for example, a USB connector) for transmitting/receiving power and/or data to/from an external electronic device, and/or a second connector hole (for example, an earphone jack) 209 capable of containing a connector for transmitting/receiving an audio signal to/from the external electronic device.

The sensor module 204, 216, or 219 may generate an electrical signal or data value corresponding to an internal operating state or an external environmental condition of the electronic device 101. Each of the sensor modules of the electronic device 101 may further include at least one of, for example, a gesture sensor, a gyro sensor, an atmospheric pressure sensor, a magnetic sensor, an acceleration sensor, a grip sensor, a color sensor, an infrared (IR) sensor, a biometric sensor, a temperature sensor, a humidity sensor, or an illuminance sensor 204.

As illustrated in the structure of FIG. 2C, each of the sensor modules 204, 216, and 219 may include an optical sensor device 210 including a light-emitting element and a light-receiving element. FIG. 2C illustrates the structure of the light sensor device 210 included in the electronic device 101 according to certain embodiments. In FIG. 2C, the side view is a view of the light sensor device 210 obtained when viewed from a side, and the top view is a view of the light sensor device 210 obtained when viewed from above.

As illustrated in FIG. 2C, the light sensor device 210 according to an embodiment may be disposed on the rear face 211B of the electronic device 101. Without being limited thereto, the light sensor device 210 may be disposed on the front face of the electronic device 101.

According to an embodiment, the light sensor device 210 may include a substrate 220, at least one light-emitting element 230, and a light-receiving element 240 disposed on the substrate 220. The light sensor device 210 may include a window 250 disposed above the at least one light-emitting element 230 and light-receiving element 240.

Light emitted from the light-emitting element 230 is transmitted or reflected, and the light sensor device 210 may sense the reflected light through the light-receiving element 240 so as to detect (e.g., acquire, generate) information related to the electronic device 101, the external environment of the electronic device 101, or the user of the electronic device. For example, the light sensor device 210 may measure the user's heart rate, oxygen saturation, or blood sugar through light reflected from the user's body.

The light-emitting element 230 may include one of a light-emitting diode (LED), a semiconductor laser (LD), a solid laser, and an infrared (IR) diode. The light-emitting diode may include at least some of a red LED, a green LED, a blue LED, and a white LED.

According to an embodiment, the light sensor device 210 may include light-emitting elements 230 that emit various wavelength bands or colors in order to measure various pieces of sensing information.

According to an embodiment, the type and number of light-emitting elements 230 or light-receiving elements 240 included in the light sensor device 210 may be changed depending on the sensing services provided by the electronic device 101. FIG. 2C illustrates that four light-emitting elements 230 and one light-receiving element 240 are illustrated, but the number and/or arrangement of the light-emitting elements 230 and the light-receiving elements 240 are not limited to the configuration illustrated in FIG. 2C.

According to an embodiment, as in the top view of FIG. 2C, the light sensor device 210 may include a red LED, a green LED, a blue LED, and an infrared (IR) LED. The blue LED may emit light in a 455-565 nm wavelength band, the green LED may emit light in a 620-635 nm wavelength band, the red LED may emit light in a 655-665 nm wavelength band, and the IR diode may emit light in a 930-955 nm wavelength band.

For example, if the light sensor device 210 is a PPG sensor device, the user's heart rate may be measured using the IR diode and the red LED or the IR diode and the green LED. The light sensor device 210 may measure the oxygen saturation of the user of the electronic device using the IR diode and the red LED. The light sensor device 210 may measure the blood sugar of the user of the electronic device using the blue LED, or may measure the blood pressure of the user of the electronic device using the green LED.

The light-receiving element 240 may measure the reflected light or incident light of the light emitted by the light-emitting elements 230. The light-receiving element 240 may include one of a photo diode (PD) and an avalanche photo diode (APD).

According to an embodiment, the light-receiving element 240 may be implemented including a single layer or including at least two layers.

According to an embodiment, the light-receiving element 240 may be implemented as an element having a response (e.g., a gain depending the wavelength) corresponding to the wavelength characteristic of the light-emitting elements 230.

According to an embodiment, in the light sensor device 210, the light-emitting elements 230 and the light-receiving element 240 may be arranged side by side on the substrate 220, but may be integrally formed.

According to an embodiment, in the light sensor device 210, a window 250 may be disposed above the light-emitting elements 230 and the light-receiving element 240, and a spacer 255 may be disposed to support the window 250. The window 250 may have a size that can simultaneously cover the light-receiving element 240 and the light-emitting elements 230. The window 250 may have a structure, at least a part of which is optically transparent. For example, the window 250 may have a structure in which the areas corresponding to the disposed locations of the light-emitting elements 230 and the light-receiving element 240 are transparent.

According to an embodiment, as illustrated in the top view, the window 250 may further include a shielding unit 257 for excluding mutual interference of the light-emitting elements 230 and the light-emitting element 240. For example, the window 250 may be black-processed in an area other than the areas corresponding to the disposed locations of the light-emitting elements 230 and the light-receiving element 240. According to an embodiment, the shielding unit may be formed of various materials, such as rubber, urethane, and PC.

According to an embodiment, the light sensor device 210 is mounted inside the electronic device 101, and a part thereof (e.g., the disposed locations of the light-emitting elements 230 and the light-receiving element 240) may be disposed so as to be disposed to the external environment through the housing (or the cover glass) of the electronic device.

The light sensor device 210 according to certain embodiments may include a pattern for improving optical efficiency in at least a part of the light sensor device 210, and may be mounted inside the electronic device using an injection-molded lens covering the light sensor device 210.

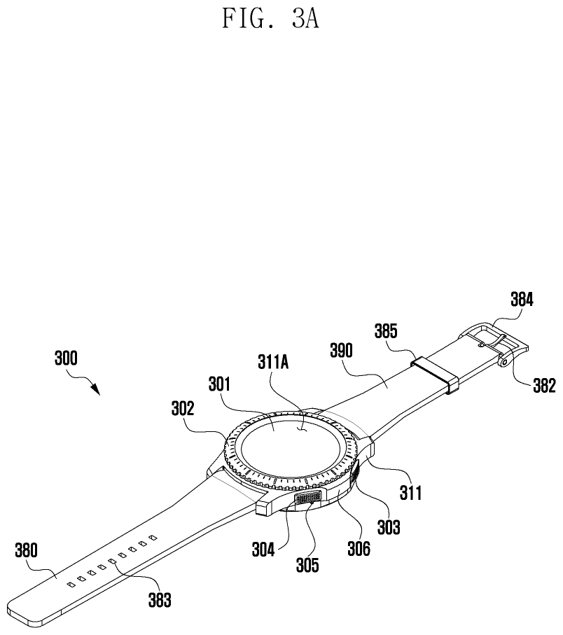

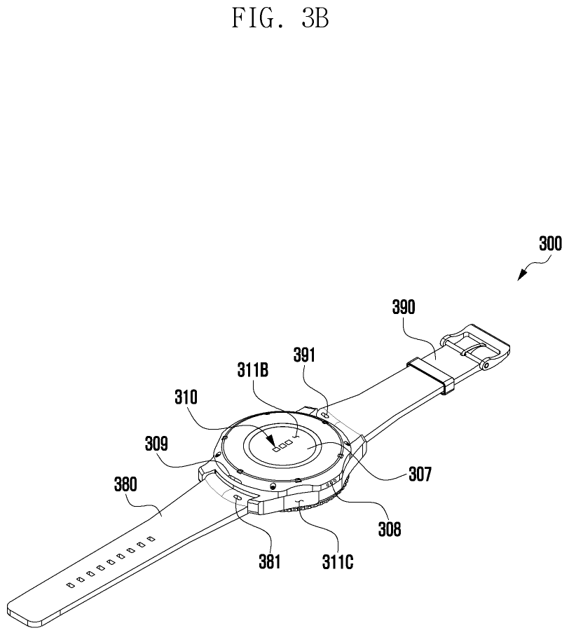

FIG. 3A is a front perspective view of an electronic device 300 according to certain embodiments, and FIG. 3B is a rear perspective view of the electronic device 300 according to certain embodiments.

Referring to FIGS. 3A and 3B, the electronic device 300 (e.g., the electronic device 101 of FIG. 1) according to an embodiment may be a wrist-wearable device (e.g., a watch-type, bracelet-type, band-type, or bangle type device) as illustrated in FIG. 3A. According to an embodiment, if the electronic device 300 is a wearable device, as illustrated in FIG. 3B, an optical sensor device 310 may be exposed on the rear face 311B of the electronic device (e.g., the face that comes into contact with the user's body when the user wears the electronic device). As illustrated in FIG. 2C described above, the light sensor device 310 may include a light-emitting element 330 and a light-receiving element 340, and may be exposed to the external environment from the rear surface 311B of the electronic device 300 so as to emit light towards an external object (e.g., such as the user's body) and to detect light reflected from the external object.

According to an embodiment, the light sensor device 310 may be a photoplethysmography (PPG) sensor device. In order to describe an example embodiment, the light sensor device 310 implemented in the electronic device 300 will be described on the basis of a photoplethysmography (PPG) sensor, but is not limited thereto. Various light sensor devices including a light-emitting element 330 and a light-receiving element may be implemented as the light sensor device 310 of the disclosure. For example, the PPG sensor may detect the light output from the light-emitting element 330 (e.g., an LED) and reflected by an external object (e.g., the user's body) using the light-receiving element 340 (e.g., a photodiode), and may measure biometric information of at least one of the user's heart rate, stress, blood oxygen saturation (SpO2), and blood sugar on the basis of the reflected light detected by the light-receiving element 340.

According to an embodiment, if the light sensor device 310 is a photoplethysmography (PPG) sensor device, an LED having N different wavelengths may be included as a light source. For example, a green wavelength light source may be used to measure the heart rate of the human body. The green wavelength light generally shallowly penetrates into the skin, and may exhibit a strong noise resistance characteristic. A red wavelength light source may be used to measure heart rate more accurately as the red wavelength light penetrates more deeply into a human skin relative to the penetration of the green wavelength light. A light source using an infrared (IR) wavelength may be used to acquire biometric information such as a heart rate and oxygen saturation (SpO2) of a human body. A blue wavelength light source may be used to measure blood sugar. If the light sensor device 310 uses red, green, and infrared wavelengths as the light source, the light sensor device 310 may further measure skin tone. The light sensor device 310 may be implemented to obtain additional types of biometric information by including light sources of various LED wavelength bands.

An electronic device 300 (e.g., the electronic device 101 in FIG. 1) according to an embodiment may include: a housing 311 including a first face (or a front face) 311A, a second face (or a rear face) 311B, and a side face 311C surrounding the space between the first face 311A and the second face 311B; and binding members 380 and 390 connected to at least a portion of the housing 311 and configured to detachably bind the electronic device 300 on a portion of a user's body (e.g., a wrist or an ankle). In another embodiment (not illustrated), the term “housing” may refer to a structure forming a part of the first face 311A, the second face 311B, and the side face 311C in FIG. 3A. According to an embodiment, at least a portion of the first face 311A may be formed of a substantially transparent front plate 301 (e.g., a glass plate or a polymer plate including various coating layers). The second face 311B may be formed by a substantially opaque rear plate 307. The rear plate 307 may be formed of, for example, coated or colored glass, ceramic, polymer, or metal (e.g., aluminum, stainless steel (STS), or magnesium), or a combination of two or more of these materials. The side face 311C may be formed by a side bezel structure (or a “side member”) 306 coupled to the front plate 301 and the rear plate 307 and including metal and/or polymer. In some embodiments, the rear plate 307 and the side bezel structure 306 may be integrally formed, and may include the same material (e.g., a metal material such as aluminum). The binding members 380 and 390 may be formed of various materials and in various shapes. A plurality of integrated-type unit links may be formed to be movable with respect to each other using a woven material, leather, rubber, urethane, metal, ceramic, or a combination thereof.

According to an embodiment, the electronic device 300 may include at least one of a display 320 (e.g., the display device 160 in FIG. 1), audio modules 305 and 308, a sensor module 310, key input devices 302, 303, and 304, and a connector hole 309. In some embodiments, in the electronic device 300, at least one of the components ((e.g., the key input devices 302, 303, and 304, the connector hole 309, or the sensor module 310) may be omitted, or other components may be added.

The display 320 (e.g., the display device 160 in FIG. 1 or a user interface) may be exposed through, for example, a first portion (e.g., a substantial portion of the front plate 301). The shape of the display 320 may have a shape corresponding to the shape of the front plate 301, and may have various shapes such as a circle, an ellipse, and a polygon. The display 320 may be coupled to or disposed adjacent to a touch sensing circuit, a pressure sensor capable of measuring touch intensity (pressure), and/or a fingerprint sensor.

An electronic device 300 (e.g., the electronic device 101 in FIG. 1) according to an embodiment may include: a housing 311 including a first face (or a front face) 311A, a second face (or a rear face) 311B, and a side face 311C surrounding the space between the first face 311A and the second face 311B; and binding members 380 and 390 connected to at least a portion of the housing 311 and configured to detachably bind the electronic device 300 on a portion of a user's body (e.g., a wrist or an ankle). In another embodiment (not illustrated), the term “housing” may refer to a structure forming a part of the first face 311A, the second face 311B, and the side face 311C in FIG. 3A. According to an embodiment, at least a portion of the first face 311A may be formed of a substantially transparent front plate 301 (e.g., a glass plate or a polymer plate including various coating layers). The second face 311B may be formed by a substantially opaque rear plate 307. The rear plate 307 may be formed of, for example, coated or colored glass, ceramic, polymer, or metal (e.g., aluminum, stainless steel (STS), or magnesium), or a combination of two or more of these materials. The side face 311C may be formed by a side bezel structure (or a “side member”) 306 coupled to the front plate 301 and the rear plate 307 and including metal and/or polymer. In some embodiments, the rear plate 307 and the side bezel structure 306 may be integrally formed, and may include usage of the same material (e.g., a metal material such as aluminum). The binding members 380 and 390 may be formed of various materials and be formed in various shapes. The binding members 380 and 390 may be formed to be movable with respect to each other using a woven material, leather, rubber, urethane, metal, ceramic, or a combination thereof.

According to an embodiment, the electronic device 300 may include at least one of a display 320 (e.g., the display device 160 in FIG. 1), audio modules 305 and 308, a sensor module 310, key input devices 302, 303, and 304, and a connector hole 309. In some embodiments, in the electronic device 300, at least one of the components ((e.g., the key input devices 302, 303, and 304, the connector hole 309, or the sensor module 310) may be omitted, or other components may be added.

The display 320 (e.g., the display device 160 in FIG. 1 or a user interface) may be exposed through, for example, a first portion (e.g., a substantial portion of the front plate 301). The shape of the display 320 may have a shape corresponding to the shape of the front plate 301, and may have various shapes such as a circle, an ellipse, and a polygon. The display 320 may be coupled to or disposed adjacent to a touch sensing circuit, a pressure sensor capable of measuring touch intensity (pressure), and/or a fingerprint sensor.

The key input devices 302, 303, and 304 may include a wheel key 302 disposed on the first face 311A of the housing 311 and configured to be rotatable in at least one direction, and/or side key buttons 302 and 303 disposed on the side face 311C of the housing 311. The wheel key may have a shape corresponding to the shape of the front plate 301. In another embodiment, the electronic device 300 may not include some or all of the above-mentioned key input devices 302, 303, and 304, and a non-included key input device 302, 303, or 304 may be implemented in another form such as a soft key on the display 320. The defined connector hole 309 may accommodate a connector (e.g., a USB connector) configured to transmit and receive power and/or data to and from an external electronic device, and may define another connector hole (not illustrated) capable of accommodating a connector configured to transmit and receive an audio signal to and from an external electronic device. The electronic device 300 may further include, for example, a connector cover (not illustrated) that covers at least a portion of the connector hole 309 and blocks entry of external foreign matter into the connector hole.

Each of the binding members 380 and 390 may be detachably fastened to at least a portion of the housing 311 using locking member 381 and 391. Each of the binding members 380 and 390 may include at least one of a fixing member 382, fixing member fastening holes 383, a band guide member 384, and a band fixing ring 385.

The fastening member 382 may be configured to secure the housing 311 and the binding members 380 and 390 to a portion of the user's body (e.g., a wrist or an ankle). The fixing member fastening holes 383 allow the housing 311 and the fastening members 380 and 390 to be secured to a portion of the user's body in cooperation with the fixing member 382. The band guide member 384 is configured to limit the movement range of the fixing member 382 when the fixing member 382 is fastened to any of the fixing member fastening holes 383 so as to ensure that the binding members 380 and 390 are brought into close contact with and bound on the user's body part. The band fixing ring 385 is capable of limiting the movement range of the binding members 380 and 390 in the state in which the fixing member 382 and a fixing member fastening holes 383 are fastened to each other.

Hereinafter, the structure of the light sensor device 210 coupled with an injection-molded lens on which a pattern for improving optical efficiency is integrally formed will be described.

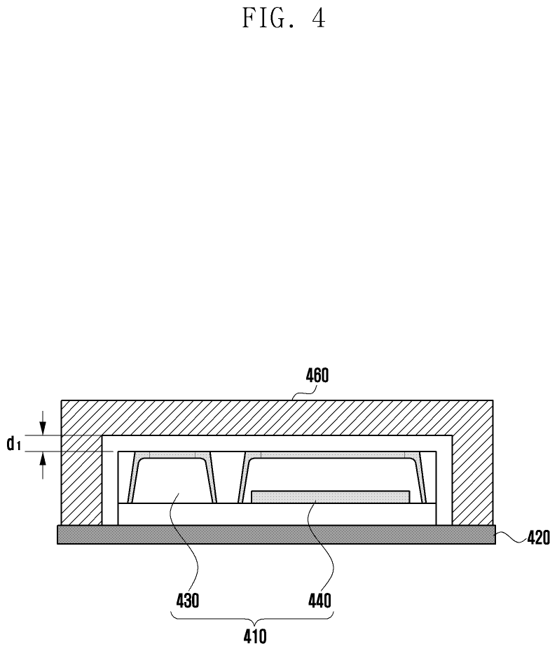

FIG. 4 is a vertical cross-sectional view illustrating an optical sensor device according to certain embodiments.

Referring to FIG. 4, an electronic device (e.g., the electronic device 101 in FIG. 1 or the electronic device 300 in FIG. 3) according to certain embodiments may include a printed circuit board (PCB) 420, an optical sensor device 410 mounted on the PCB 420, and an injection-molded lens 460 attached to the PCB 420 as a structure covering the light sensor device 410.

The PCB 420 may include at least one element or circuit pattern (not illustrated) for driving the electronic device. The PCB 420 may be formed of a flexible printed circuit board (FPCB), but is not limited thereto.

According to an embodiment, the light sensor device 410 may be mounted on the PCB 420 with the structure of the light sensor device 210 of FIG. 2C, but is not limited thereto. For example, the light sensor device 410 may include at least one light-emitting element 430 and a light-receiving element 440. The light sensor device 410 of FIG. 4 is schematically illustrated for convenience of description, and may further include a window (e.g., the window 250 in FIG. 2C) disposed above the light-emitting element 430 and the light-receiving element 440, a spacer that supports the window (e.g., the spacer 255 in FIG. 2C), and a shield member (e.g., the shield member 257 in FIG. 2C). According to an embodiment, the light sensor device 410 may be mounted on the PCB 420 through surface mount technology (SMT).

According to an embodiment, the injection-molded lens 460 is spaced apart from the light sensor device 410 by a set distance “dl” (as illustrated) and disposed on the PCB 420 with a structure covering the entire light sensor device 410. For example, the injection-molded lens 460 may include a top part positioned above the light sensor device 410, and a side part positioned on the side of the light sensor device 410 to support the top part. The terms “top part” and “side part” are used merely for differentiation in describing the disclosure, and the injection-molded lens 460 may be an integral structure including the top part and the side part.

According to an embodiment, the bottom face of the top part of the injection-molded lens 460 may be disposed to be spaced apart from the top face of the light sensor device 410 by a predetermined distance dl. The distance dl may be, for example, 0.2 mm, but may be changed without being limited thereto.

According to an embodiment, the injection-molded lens 460 may include a pattern for improving optical efficiency, which is integrally formed on at least one face thereof corresponding to the light sensor device 410, and may be formed to have an opaque color capable of selectively transmitting at least a part of light. Due to the pattern and color of the injection-molded lens 460, the light sensor device 410 is capable of visually blocking the light-emitting element 430 and the light-receiving element 440 therein while being improved in optical efficiency.

According to an embodiment, the injection-molded lens 460 may have various types of patterns implemented on at least one of the top and bottom faces of the top part by an injection mold. For example, if an upper mold is formed such that a specific pattern is formed on the top face of the top part of the injection-molded lens 460 and the upper mold is coupled with a lower mold, the injection-molded lens 460 may be manufactured by pouring a resin material into the coupled molds and curing the resin material. Here, the upper mold or the lower mold may be a mold, which is implemented such that one face of the top part transmitting light has a specific pattern.

According to an embodiment, the injection-molded lens 460 may be, but is not limited to, a prism lens. The injection-molded lens 460 may include at least one of polymethylmethacrylate (PMMA), polycarbonate (PC) or polyethylene (PE). According to an embodiment, the injection-molded lens 460 may be formed by changing the color of the lens differently depending on the emission wavelength of the light-emitting element 430 mounted on the light sensor device 410.

According to an embodiment, the injection-molded lens 460 may be formed to integrally implement patterns in various shapes such that light may be transmitted or reflected at a desired angle.

According to an embodiment, although not illustrated, the injection-molded lens 460 may be attached to the PCB 420 using one of double-sided tape, or a bonding or ultrasonic welding method. For example, the injection-molded lens 460 may be fixed in such a manner that the bottom face thereof is in contact with the PCB 420 using double-sided tape (not illustrated) along the edge of the bottom face of the side part.

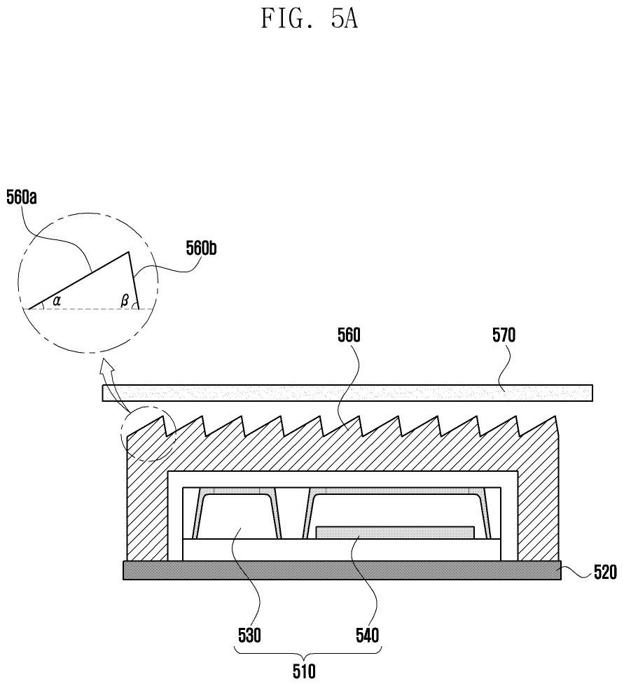

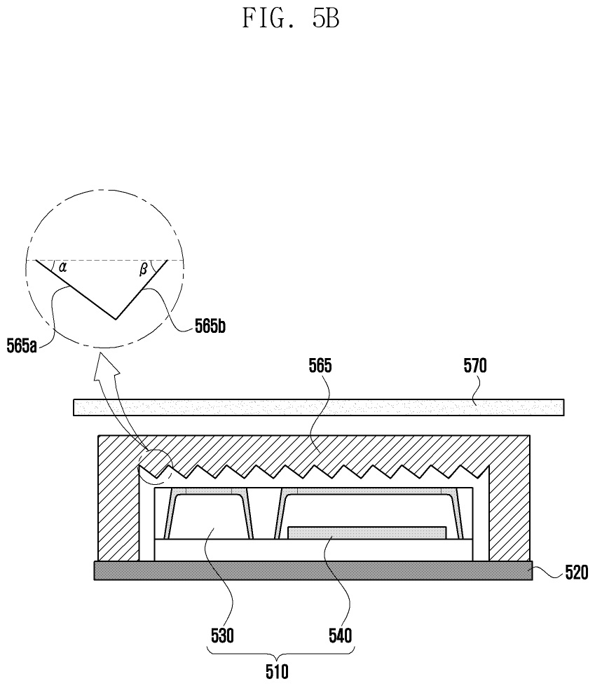

FIG. 5A is a vertical cross-sectional view illustrating an optical sensor device according to certain embodiments, and FIG. 5B is a vertical cross-sectional view illustrating an optical sensor device according to certain embodiments.

Referring to FIGS. 5A and 5B, an electronic device (e.g., the electronic device 101 in FIG. 1 or the electronic device 300 in FIG. 3) according to certain embodiments may include a PCB 520, an optical sensor device 510 mounted on the PCB 520, an injection-molded lens 560 attached to the PCB 520 as a structure that covers the light sensor device 510, and a housing 570 of the electronic device (e.g., a window or a cover glass) disposed above the injection-molded lens 560. As illustrated in FIG. 4, the light sensor device 510 may include a light-emitting element 530 and a light-receiving element 540. Since the detailed configuration of the sensor device 510 is substantially the same as those described above with reference to FIGS. 2C and 4, a repetitive description of these elements will be omitted for the sake of brevity.

According to an embodiment, the injection-molded lens 560 may have a pattern for improving optical efficiency, which is integrally formed on at least one face thereof. Integrally formed patterns may be implemented in various shapes or in various angles depending on the type of the light sensor device 510 mounted in the electronic device or the light wavelength of the light-emitting element 530 included in the light sensor device 510.

As illustrated in FIG. 5A, the injection-molded lens 560 may be formed as a structure in which first patterns are integrally formed on the top face of the top part disposed above the light sensor device 510. For example, the first patterns may be formed in the shape of a saw blade having first faces 560a and second faces 560b on the top face of the top part, such that the first faces 560a are disposed at an angle α of approximately 30 degrees with respect to the horizontal direction, and the second faces 560b may be formed to have an angle β of approximately 60 degrees with respect to the horizontal direction. The injection-molded lens 560 may include the patterns formed to have the angles as illustrated in FIG. 5A, which may provide the effect of increasing the amount of light by approximately 139% to 165% generally in the infrared, red, green, and blue wavelength bands. If the light sensor device 510 includes the light-emitting element 530 having infrared, red, green, and blue wavelength bands, the injection-molded lens 560 having the same structure as the first patterns illustrated in FIG. 5A may be disposed together with the light sensor device 510 to improve the effect of increasing the amount of light.

As illustrated in FIG. 5B, the injection-molded lens 565 may be formed as a structure in which second patterns are integrally formed on the bottom face of the top part disposed above the light sensor device 510. For example, the second patterns may be formed in the shape of a saw blade having first faces 565a and second faces 565b disposed on the bottom face of the top part, such that the first faces 565a have an angle α of approximately 30 degrees with respect to the horizontal direction and the second faces 565b may be formed to have an angle β of approximately 40 degrees with respect to the horizontal direction. The injection molded lens 565 including the patterns having the angles as illustrated in FIG. 5B may provide the effect of increasing the amount of light by approximately 205% to 230% intensity in the blue and red wavelength bands. For example, if the light sensor device 510 includes the light-emitting element 530 having blue and red wavelengths, it is possible to improve the effect of selectively increasing the amount of light for a particular wavelength band by disposing the injection-molded lens 565 having the same structure as the second patterns illustrated in FIG. 5B together with the light sensor device 510.

According to an embodiment, the housing 570 of the electronic device may be a front plate or a back plate of the electronic device. The housing may be formed of coated or colored glass, ceramic, polymer, metal (e.g., aluminum, stainless steel (STS), or magnesium), or a combination of at least two of the above materials, and at least a portion of the housing may have a substantially transparent structure. For example, a front plate, a rear plate, and a side plate may be coupled to the housing 570 of the electronic device so as to mount electronic components implementing the electronic device.

According to an embodiment, the light sensor device 510 may be disposed under the rear plate of the electronic device or under the front plate. Alternatively, the light sensor device 510 may be disposed under the side plate of the electronic device.

According to certain embodiments, an electronic device (e.g., the electronic device 101 in FIG. 1 or the electronic device 300 in FIG. 3) may include: a substrate (e.g., the PCB 420 in FIG. 3 or the PCB 520 in FIGS. 5A and 5B); an optical sensor device (e.g., the light sensor device 210 in FIG. 2C, the light sensor device 410 in FIG. 4, or the light sensor device 510 in FIGS. 5A and 5B) including at least one light-emitting element and a light-receiving element and mounted on the substrate; and an injection-molded lens (e.g., the injection-molded lens 460 in FIG. 4, the injection-molded lens 560 in FIG. 5A, or the injection-molded lens 565 in FIG. 5B) attached to the substrate as a structure that covers the light sensor device in the state of being spaced apart from the light sensor device by a set distance. The injection-molded lens may have patterns integrally formed in at least a portion thereof to transmit light having at least one wavelength band and to improve the optical efficiency of the transmitted light.

In some embodiments, the injection-molded lens may include a top part disposed above a top face of the light sensor device and a side part for supporting the top part as a side face of the light sensor device, and the top part and the side part may be integrally formed.

In some embodiments, the patterns may be formed in at least one of a top face and a bottom face of the top part of the injection-molded lens.

In some embodiments, the injection molded lens may be formed to have an opaque color capable of selectively transmitting at least a part of the light.

In some embodiments, the injection-molded lens may be formed to have a color determined to correspond to light having a wavelength band of the at least one light-emitting element included in the light sensor device.

In some embodiments, the injection-molded lens may include a prism lens.

In some embodiments, the patterns formed on at least one of the top face and the bottom face of the top part of the injection-molded lens may include a saw-blade structure, and the saw-blade structure may be formed to have different angles depending on the light wavelength band of the light-emitting element.

In some embodiments, the patterns formed on at least one of the top face and the bottom face of the top part of the injection molded lens may be determined as patterns, which improve optical efficiency depending on the light wavelength band of the at least one light-emitting device included in the optical sensor device.

In some embodiments, if the light-emitting element of the light sensor device includes a diode of a first wavelength band, first patterns may be integrally formed in at least a portion of the injection-molded lens, and if the light-emitting element of light sensor device includes a diode of a second wavelength band, second patterns may be integrally formed in at least a portion of the injection-molded lens.

If the at least one light-emitting element includes a diode of a first wavelength band and a diode of a second wavelength band, first patterns are integrally formed in an area in which the diode of the first wavelength band is located, and second patterns are integrally formed in an area in which the diode of the second wavelength band is located.

In some embodiments, the injection-molded lens may have first patterns formed in an area in which the at least one light-emitting element is located and second patterns formed in an area in which the light-receiving element is located.

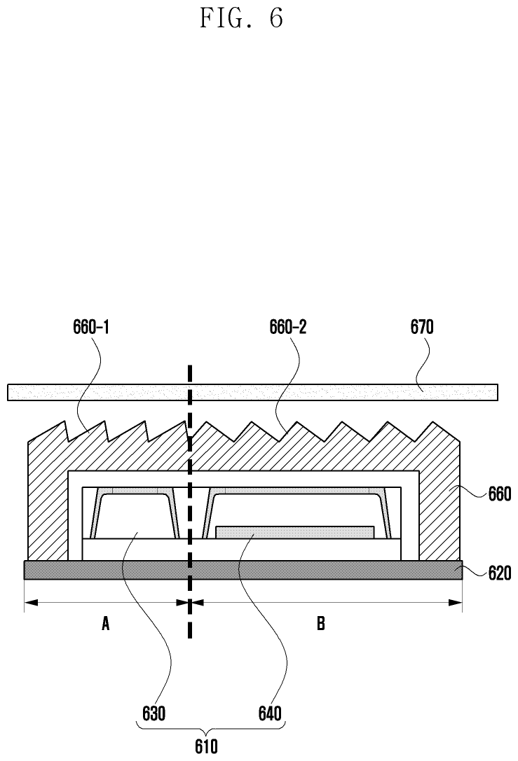

FIG. 6 is a vertical cross-sectional view illustrating an optical sensor device according to certain embodiments.

Referring to FIG. 6, an electronic device (e.g., the electronic device 101 in FIG. 1 or the electronic device 300 in FIG. 3) according to certain embodiments may include a PCB 620, an optical sensor device 610 including a light-emitting area A and a light-receiving area B and mounted on the PCB 620, an injection-molded lens 660 configured to cover the light sensor device 610 and having a first pattern 660-1 formed in the light-emitting area A and a second pattern 660-2 formed in the light-receiving area B, and a housing 670 of the electronic device (e.g., a window or a cover glass) disposed above the injection-molded lens 660.

According to an embodiment, in the light-emitting area A of the light sensor device 610, at least one light-emitting element 630 may be disposed, and in the light-receiving area B, a light-receiving element 640 may be disposed. Since the detailed configuration is substantially the same as those illustrated in FIGS. 2 and 4, a description thereof will be omitted.

According to an embodiment, in the injection-molded lens 660, the first pattern 660-1 for improving the optical efficiency of the light emitted from the light-emitting element 630 may be formed at a position corresponding to the light-emitting region A, and the second pattern 660-2 for suppressing the loss of reflected light may be formed at a position corresponding to the light-receiving area B.

Although FIG. 6 illustrates that the first pattern 660-1 and the second pattern 660-2 of the injection-molded lens 660 are formed on the top face of the top part of the injection-molded lens 660, in some embodiments, the first pattern 660-1 and the second pattern 660-2 of the injection-molded lens 660 may be formed on the bottom face of the top part. In some embodiments, the first pattern 660-1 of the injection-molded lens 660 may be formed on the top face of the top part in the light-emitting area A, and the second pattern 660-2 may be formed on the bottom face of the top part in the light-receiving area B.

In some embodiments, in the light sensor device 610, a plurality of light-emitting elements 630 having different wavelength bands may be disposed in the light-emitting area A. Although FIG. 6 illustrates that a single first pattern is formed in the light-emitting area, a plurality of patterns may be formed in a complex form by dividing the light-emitting area.

For example, in the light sensor device 610, the light-emitting area A is divided into four portions, and a red LED may be disposed in a first zone, a green LED may be disposed in a second zone, a blue LED may be disposed in a third zone, and an IR LED may be disposed in a fourth zone. In this case, a pattern for improving the optical efficiency of the red LED may be formed in the first zone of the light-emitting area A of the injection-molded lens 660, patterns for improving the optical efficiency of the green LED may be formed in the second zone, patterns for improving the optical efficiency of the blue LED may be formed in the third zone, and patterns for improving the optical efficiency of the IR LED may be formed in the fourth zone. The patterns formed integrally with the injection-molded lens 660 may be formed differently depending on the light wavelength of the light-emitting element 630. In some embodiments, if the optical sensor device 610 includes the light-emitting elements 630 of different wavelength bands, the optical sensor device 610 may further include a shield unit for suppressing interference to respective light-emitting elements 630.

For example, if saw blade-shaped patterns are formed on the top face of the top part of the injection-molded lens 660 and if the first faces of the saw blade are 30 degrees and the second faces are 70 degrees in the first zone where the red LED is disposed, the maximum amount of light may be increased by 170%. In the second zone in which the green LED is disposed, the maximum amount of light may be increased by 143% if the first faces of the saw blade are 30 degrees and the second faces are 80 degrees. In the third zone in which the blue LED is disposed, the maximum amount of light may be increased by 165% if the first faces of the saw blade are 30 degrees and the second faces are 80 degrees. In the fourth zone in which the IR LED is disposed, the maximum amount of light may be increased by 157% if the first faces of the saw blade are 30 degrees and the second faces are 40 degrees.

According to an embodiment, a plurality of light sensor devices 610 may be installed at various locations of the electronic device. For example, one HRM sensor device may be disposed on the rear face of the electronic device, and the light sensor devices may be additionally or alternately disposed on the side face of the electronic device or the front face of the electronic device.

For example, a front plate, a rear plate, and a side plate may be coupled to the housing 670 of the electronic device so as to mount electronic components constituting the electronic device. According to an embodiment, the light sensor device 610 may be disposed under the rear plate of the electronic device or under the front plate. Alternatively, the light sensor device 610 may be disposed under the side plate of the electronic device.

According to certain embodiments, an electronic device (e.g., the electronic device 101 in FIG. 1) may include: a substrate (e.g., the PCB 420 in FIG. 4, the PCB in FIGS. 5A and 5B, or the PCB 620 in FIG. 6); an optical sensor device (e.g., the light sensor device 210 in FIG. 2C, the light sensor device 410 in FIG. 4, the light sensor 510 in FIGS. 5A and 5B, or the light sensor device 610 in FIG. 6) including at least one light-emitting element and a light-receiving element and mounted on the substrate; and an injection-molded lens (e.g., the injection-molded lens 460 in FIG. 4, the injection-molded element 560 in FIG. 5A, the injection-molded lens 565 in FIG. 5B, or the injection-molded lens 660 in FIG. 6) attached to the substrate as a structure that covers the light sensor device in a state of being spaced apart from the light sensor device by a set distance. The injection-molded lens may include a top part disposed above a top face of the light sensor device and a side part for supporting the top part as a side face of the light sensor device, and patterns may be integrally formed in at least a portion of the injection-molded lens to transmit light having at least one wavelength band and to improve the optical efficiency of the light.

According to certain embodiments disclosed herein, the electronic device may include at least one electronic component exposed through a portion of the housing, and the HRM sensor may be disposed adjacent to the electronic component.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Certain embodiments as set forth herein may be implemented as software (e.g., the program 140) including one or more instructions that are stored in a storage medium (e.g., internal memory 136 or external memory 138) that is readable by a machine (e.g., the electronic device #01). For example, a processor(e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. The term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to certain embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

What is claimed is:

1. An electronic device, comprising:
a substrate;
an optical sensor device including at least one light-emitting element and a light-receiving element, the optical sensor device mounted on the substrate; and
an injection-molded lens coupled to the substrate and covering the optical sensor device, wherein the injection-molded lens is spaced apart from the optical sensor device by a set distance,
wherein patterns are integrally formed in at least a portion of the injection-molded lens, the patterns affecting transmission of light of at least one wavelength band to improve an optical efficiency of the transmitted light.

2. The electronic device of claim 1, wherein the injection-molded lens includes a top part disposed above a top face of the optical sensor device, and a side part supporting the top part as a side face of the optical sensor device, wherein the top part and the side part are integrally formed.

3. The electronic device of claim 2, wherein the patterns are formed in at least one of a top face and a bottom face of the top part of the injection-molded lens.

4. The electronic device of claim 2, wherein the injection-molded lens includes an opaque color which selectively transmits at least a part of the transmitted light.

5. The electronic device of claim 4, wherein the injection-molded includes a color corresponding to a wavelength band of the at least one light-emitting element included in the optical sensor device.

6. The electronic device of claim 4, wherein the injection-molded lens includes a prism lens.

7. The electronic device of claim 3, wherein the patterns formed on at least one of the top face and the bottom face of the top part of the injection-molded lens include a saw-blade structure including a plurality of ridges, wherein the ridges are set at different angles relative to one another according to the at least one light wavelength band of the light-emitting element.

8. The electronic device of claim 3, wherein the patterns improve the optical efficiency of the transmitted light depending on the light wavelength band of the at least one light-emitting element included in the optical sensor device.

9. The electronic device of claim 3, wherein the light-emitting element includes at least one of a first diode that emits light of a first wavelength band, and a second diode that emits light of a second wavelength band, and
wherein the injection-molded lens includes at least one of a first set of patterns for enhancing transmission of the light of the first wavelength band, and a second set of patterns for enhancing transmission of the light of the second wavelength band.

10. The electronic device of claim 9, wherein the first set of patterns are formed in a first area of the injection-molded lens corresponding with disposition of the first diode, and the second set of patterns are formed in a second area of the injection-molded lens corresponding with disposition of the second diode.

11. The electronic device of claim 3, wherein the injection-molded lens includes first patterns formed in an area in which the at least one light-emitting element is located, and second patterns formed in an area in which the light-receiving element is located.

12. An electronic device, comprising:
a circuit board including a substrate;
an optical sensor device including at least one light-emitting element and a light-receiving element, the optical sensor device mounted on the substrate; and
an injection-molded lens coupled to the substrate and covering the optical sensor device, wherein the injection-molded lens is spaced apart from the optical sensor device by a set distance,
wherein the injection-molded lens includes a top part disposed above a top face of the optical sensor device, and a side part supporting the top part as a side face of the optical sensor device, and patterns are integrally formed in at least a portion of the injection-molded lens, the patterns affection transmission of light of at least one wavelength band to improve an optical efficiency of the transmitted light.

13. The electronic device of claim 12, wherein the injection-molded lens includes a prism lens having an opaque color.

14. The electronic device of claim 13, wherein the patterns are formed on at least one of the top face and the bottom face of the top part of the injection-molded lens, and
wherein each of the patterns are differentiated according to each respective wavelength band of a plurality of light-emitting elements included in the optical sensor device.

15. The electronic device of claim 13, wherein the patterns improve the optical efficiency of the transmitted light depending on the light wavelength band of the at least one light-emitting element included in the optical sensor device.

16. The electronic device of claim 13, wherein the light-emitting element includes at least one of a first diode that emits light of a first wavelength band, and a second diode that emits light of a second wavelength band, and
wherein the injection-molded lens includes at least one of a first set of patterns for enhancing transmission of the light of the first wavelength band, and a second set of patterns for enhancing transmission of the light of the second wavelength band.

17. The electronic device of claim 16, wherein the first set of patterns are formed in a first area of the injection-molded lens corresponding with disposition of the first diode, and the second set of patterns are formed in a second area of the injection-molded-lens corresponding with disposition of the second diode.

18. The electronic device of claim 13, wherein the injection-molded lens includes first patterns formed in an area in which the at least one light-emitting element is located, and second patterns formed in an area in which the light-receiving element is located.
